(19) United States
(12) Patent Application Publication
(10) Pub. No.: US 2007/0224087 A1
(43) Pub. Date: Sep. 27, 2007

(54) Airborne material collection and detection method and apparatus

(76) Inventor: Zhong Ding, Pittsford, NY (US)

Correspondence Address: ZHONG DING, 35 DELANCEY COURT, PITTSFORD, NY, 14534, US

(21) Appl. No.: 11/176,176

(22) Filed: Jul. 7, 2005

Related U.S. Application Data
(60) Provisional application No. 60/586,203, filed on Jul. 8, 2004.
(60) Provisional application No. 60/663,963, filed on Mar. 21, 2005.

(51) Int. Cl. G01N 33/00

(52) U.S. Cl. 422/83

(57) ABSTRACT

An apparatus and methods for airborne or gas borne chemical and biological sample collection and detection in real time using optical or chemical techniques, the apparatus comprising a chamber for delivery of said flowing gas stream containing a substance, the chamber comprising a first wall including a first wall surface and a longitudinal inlet channel disposed within the first wall and terminating at an outlet edge at the first wall surface; a second wall including a second wall surface and a longitudinal outlet channel disposed within the second wall and beginning at an outlet edge at the second wall surface, the longitudinal inlet channel aligned with the longitudinal outlet channel, and the first wall surface separated from the second wall surface by a gap; wherein the longitudinal inlet channel the longitudinal outlet channel are adapted for delivering a liquid into the inlet channel, through the chamber, and out the outlet channel such that a gas-liquid interface may be formed in the gap of the chamber.

### CROSS-REFERENCE TO RELATED PATENT APPLICATIONS

This application claims the benefit of the filing date of U.S. provisional patent application Ser. No. 60/586,203 filed Jul. 8, 2004, and U.S. provisional patent application Ser. No. 60/663,963 filed Mar. 21, 2005, the disclosures of which are incorporated herein by reference.

The invention relates to the collection and analysis of airborne hazardous contaminants, and more specifically to airborne chemical and biological sample collection and detection in real time with optical or chemical means.

### BACKGROUND OF THE INVENTION

1. Field of the Invention

An apparatus and methods for airborne chemical and biological sample collection and detection in real time using optical or chemical techniques.

2. Description of Related Art

Airborne hazardous contaminants are materials that may exist in the form of gaseous, aerosol, liquid, solid, or partially solid. They are very small and can be dispersed in the air and carried along with the air flow. The hazardous contaminants can be organic or inorganic chemicals. Some hazardous contaminants are microbes, i.e. bacteria, spores, viruses, and the like.

In this era of heightened homeland security, it is critical to health and safety of the public to be able to monitor for the presence of airborne hazardous contaminants continuously, and to analyze and identify such contaminants in real time, especially in the circumstances where chemical and/or biological agents may be present in a battlefield, or during a terrorist attack. It is very desirable to have an integrated sample collection and analysis device that can continuously monitor and indicate the existence of a life threatening substance in the environment in real time with low cost.

One typical class of airborne hazardous contaminant collection methods is typically referred to as “impactors,” as is described in U.S. Pat. Nos. 5,437,198 and 5,693,895, the disclosures of which are incorporated herein by reference. In these methods, air is forced to flow through a specially designed flow path with abrupt turns. The hazardous contaminants with larger mass are trapped on a solid surface in the stagnation region in the flow path due to their larger inertia. A liquid is then applied to collect the trapped hazardous contaminants for analysis. A major disadvantage of this method is that only higher density hazardous contaminants can be effectively trapped, and other materials of low inertia, such as toxic gas or mist, can not be efficiently collected. Another disadvantage of this method is that hazardous contaminants can bounce in the impactor devices without collection.

An improved method called “virtual impactor” is also available as described in U.S. Pat. Nos. 6,698,592 and 6,695,146, the disclosures of which are incorporated herein by reference. This method redirects air into two different flow streams, differentiated according to their mass. Again, lower density airborne hazardous contaminants can not be effectively collected, and hazardous contaminants can still deposit on various surfaces of virtual impactor structures, especially at curved portions.

The above two types of methods require addition of liquid to dissolve the trapped hazardous contaminants for later analysis. The big disadvantage of such two stage sample collections and analyses is that they are inefficient and it is impossible to perform detection in real time, i.e. a time frame on the order of a few minutes. Manufacturers of commercial two stage sample collection and analysis systems may indicate that their systems provide results in “real time,” but the actual detection results are provided in time frames on the order of about a half hour or more. During this time period, the concentration of such hazardous materials has reached a toxic level for a sufficient period of time such that harm has occurred to humans and other species of life that are present—i.e. “the damage has already been done.” In addition, to attempt to adapt such systems to perform continuous collection, wash, and analysis of an air stream would likely require a system that is too high in cost.

U.S. Pat. No. 6,729,196, the disclosure of which is incorporated herein by reference, teaches the combining of the impact collector and a fan to collect the particulate. The captured particulate is rinsed from the combined impact collector and fan with rinsing liquid stored in the device. The rinsing liquid is then collected for analysis. One of the disadvantages of this method is that particulate in the collector and fan can not be rinsed efficiently by the liquid. Another disadvantage is that smaller particulate, especially those of gaseous phase or aerogel, can not be collected by the impact collector and the fan. Yet another disadvantage is the uncertainty of when to rinse. The rinse liquid can be quickly used up without any real detection if the rinse frequency is too high. Otherwise, if the time interval for rinse and analysis is too large, the airborne hazardous contaminant may have already created environmental damage, or have had an adverse effect on humans and/or other species in the environment before analysis is performed.

The conventional air samplers such as that described in U.S. Pat. No. 6,532,835, the disclosure of which is incorporated herein by reference, blow air to a liquid film on a solid surface and collect the particulates stripped from the air. The liquid is then collected for analysis. One disadvantage of this type of method is that a large volume of liquid is required for the analysis. Yet another disadvantage of the method is that it works only when a human operator initiates it based upon his judgment of a situation. The apparatus used in such method cannot be mounted on a wall to collect and monitor the airborne hazardous contaminants continuously for a long period of time due to significant evaporation of the liquid thin film created in the apparatus.

Accordingly, embodiments of the present invention are provided that meet at least one or more of the following objects of the present invention:

It is an object of this invention to provide a low cost, sensitive, continuous and automatic methods to collect airborne hazardous contaminants efficiently for real time analysis.

It is a further object of this invention to provide such a collection apparatus that can be mounted inside or outside a building.

It is a further object of this invention to provide a collection apparatus that can capture many different types of airborne hazardous contaminants using a single device, for subsequent analysis; such contaminants including but not limited to gaseous, solid, liquid, aerosol, organic, inorganic, hydrophobic, hydrophilic, radioactive, living, and non-living contaminants.

It is a further object of this invention to provide such a collection apparatus that can operate with minimum human interactions.

### SUMMARY OF THE INVENTION

In accordance with the present invention, there is provided an apparatus for analysis of a flowing gas stream containing a substance, said apparatus comprising a chamber for delivery of said flowing gas stream containing said substance, said chamber comprising a first wall including a wall surface and a first channel disposed within said first wall and terminating at an outlet edge at said wall surface, said channel provided for delivering a liquid to said chamber such that a gas-liquid interface is formed in said chamber at said outlet edge of said channel.

In accordance with the present invention, there is further provided an apparatus for analysis of a flowing gas stream containing a substance, said apparatus comprising a chamber for delivery of said flowing gas stream containing said substance, said chamber comprising a first wall including a first wall surface and a longitudinal inlet channel disposed within said first wall and terminating at an outlet edge at said first wall surface; a second wall including a second wall surface and a longitudinal outlet channel disposed within said second wall and beginning at an outlet edge at said second wall surface, said longitudinal inlet channel aligned with said longitudinal outlet channel, and said first wall surface separated from said second wall surface by a gap; wherein said longitudinal inlet channel and said longitudinal outlet channel are adapted for delivering a liquid into said inlet channel, through said chamber, and out said outlet channel such that a gas-liquid interface may be formed in said gap of said chamber.

In accordance with the present invention, there is further provided an apparatus for analysis of a flowing gas stream containing a substance, said apparatus comprising a first wall including a first hydrophobic surface upon which is disposed a first hydrophilic surface; and a second wall including a second hydrophobic surface upon which is disposed a second hydrophilic surface, said first hydrophilic surface aligned with said second hydrophilic surface, and said first hydrophilic surface separated from said second hydrophilic surface by a gap.

In accordance with the present invention, there is further provided a method for using liquid drops to capture gas borne substances in a flowing gas stream, said method comprising the steps of providing an apparatus comprising a chamber for delivery of said flowing gas stream containing said substance, said chamber comprising a first wall including a first wall surface, a second wall including a second wall surface separated from said first wall surface by a gap; causing said flowing gas stream to flow though said chamber; providing a liquid that forms at least one gas-liquid interface in said chamber in said gap between said first wall surface and said second wall surface; detecting at least one substance in said liquid that has passed from said flowing gas stream through said gas-liquid interface into said liquid.

In accordance with the present invention, there is further provided a method for using a liquid to capture gas borne substances in a flowing gas stream, said method comprising the steps of exposing at least part of said liquid to said gas stream to form a gas-liquid interface; limiting the area of each said gas-liquid interface so that said gas-liquid interface is stabilized by surface tension; and maintaining said gas-liquid interface in a fixed position on a solid surface.

In the present invention, tiny liquid drops or other liquid forms comprised of small free surfaces that are stabilized by surface tension are created on a solid surface to continuously collect airborne hazardous contaminants from the air. The liquid drops are made to be either hydrophobic to capture hydrophobic airborne hazardous contaminants or hydrophilic to capture hydrophilic airborne hazardous contaminants. In various embodiment of the invention, the liquid drops are created by displacing liquid to an outlet of a channel or a capillary tube by a pump, by the force of gravity, or by capillary forces. Air containing potential contaminants is delivered by a fan or by electric potential to flow between or over the small liquid drops or liquid free surfaces formed on solid surfaces. The bulk liquid drops capture the airborne hazardous contaminant from the flowing air through the free surface.

In another embodiment of the invention, a liquid drop or plurality of drops are generated via condensation by lowering the temperature of the solid collection surface, and providing such surface with specific properties. For example, providing a hydrophilic surface surrounded by a hydrophobic surface will enable the collection and/or containment of an aqueous droplet on the hydrophilic surface. In like manner, providing a hydrophobic surface surrounded by a hydrophilic surface will enable the collection and/or containment of a hydrophobic (e.g. organic oil based) droplet on the hydrophilic surface. In general, the particular surface where it is desired to condense a droplet is chosen such that the surface has an affinity for the gaseous phase material that is desired to be condensed out of the gas stream. If it is desired to condense out an aqueous liquid phase, hydrophilic surfaces are provided. If it is desired to condense out an organic liquid phase, hydrophobic surfaces are provided. If both organic and aqueous vapor phases may be present in the flowing gas stream, both hydrophobic and hydrophilic surfaces may be provided, with condensation at each surface occurring of the particular liquid having an affinity for the particular surface.

For example, in an embodiment where it is desired to detect the presence of methane, an organic oil, e.g. olive oil, may be used as a droplet medium to capture the methane in the flowing gas stream for analysis. In such an embodiment, the oil droplet formation and maintenance would occur on a hydrophobic surface surrounded by a hydrophilic surface.

In yet another embodiment of the invention, the liquid drops are formed at small holes of a channel and stabilized by the hydrophobic surface of the solid, the surface tension of the liquid and the pressure inside the channel. Liquid flows inside the channel to carry the captured the airborne hazardous contaminants for further analysis within such channel, or to a destination downstream from such channel. The contents in the liquid drops are analyzed continuously in real time with optical density at specified wavelengths and/or optical spectral analysis methods. Multiple detections of the same or different airborne hazardous contaminants can be performed simultaneously.

In embodiments of the present invention, the collected hazardous contaminants and the liquid drops may also be analyzed with chemistry and/or biochemistry methods. The solid structures holding the liquid drops in collecting airborne hazardous contaminant can either be disposable or washable for reuse.

In one embodiment of the invention, the stability of a hydrophilic liquid drop is maintained by the hydrophobic solid surface with a hydrophilic seed (i.e. a small hydrophilic area on a surface) and the gap size between adjacent solid surfaces. The efficiency of capturing airborne hazardous contaminant is increased by arranging liquid drops in a way to increase the stagnation and vortex in the air stream. Similarly, the stability of a hydrophobic liquid drop may be maintained by the hydrophilic solid surface with a hydrophobic seed surface.

In one embodiment of the invention, the liquid drops maintained between two solid surfaces are used as the optical medium for the hazardous contaminant detection. Light directed to one end of a liquid drop passes through the liquid drop and is detected by an optical sensor at the other end of the liquid drop. The optical path through a liquid drop is determined by the geometric distance between the two opposite solid surfaces holding the liquid drop.

In another embodiment of the invention, the scattered light (or reflected light) from a liquid drop is used for optical detection of the captured airborne hazardous contaminants. In this embodiment, liquid drops attach to only one solid surface. The incident light directed at a first angle is reflected (or scattered) back to an optical sensor at a second angle. Multiple liquid drops and multiple optical sensors may share the same light source in this embodiment.

In one embodiment of the invention, when airborne hazardous contaminant is captured by the liquid drop, the change in the optical density at specified wavelength and/or optical spectral in the liquid drop is detected and analyzed.

In one embodiment, the size of the liquid drop is maintained by condensation or evaporation via temperature control of the solid surfaces.

In another embodiment of the present invention, small channels are connected to the solid surface carrying fresh liquid supply to form liquid drops between hydrophobic solid surfaces. The size of the liquid drop is controlled precisely by dispensing or withdrawing a specified amount of liquid onto (or from) the solid surface through a channel.

In one embodiment, liquid drops are removed from the solid surface by fast air flow driven by a fan, after such drops have been analyzed.

Another embodiment of the present invention includes a capillary to collect liquid drops with potential airborne hazardous contaminants for further analysis and detection. The liquid drops are removed to a specific location for optical detection or to mix and react with reagent for specific assay analysis.

In one embodiment of the present invention, special chemicals are coated with hydrophilic seeds to react with the airborne hazardous contaminant and to generate detectable signals (such as optical absorbance, chemistry luminescence, electrical conductivity, surface tension, viscosity, etc) for easy detection. A wash fluid can be supplied for certain detection assays, such as solid phase Enzyme Linked Immunosorbent Assay (ELISA).

In one embodiment, the contaminant collection liquid is provided with specific chemical reagents or rheological or surface tension properties to increase the affinity between the liquid and the airborne hazardous contaminant. For example, the mucus inside an animal's nose can be used as collecting liquid. The liquid viscosity can be increased to help stabilize the liquid drop.

In one embodiment, an electric potential is applied to the liquid drops to facilitate the capturing of charged airborne hazardous contaminants. In one embodiment, a filter is used to filter out large particles from the contaminant containing air stream, such as hair, fiber, or coarse dusts. In another embodiment, collected liquid is evaporated with heat or ultrasound if no airborne hazardous contaminants are detected. In another embodiment, a micro-refrigerator may be provided as part of the system for temperature control of the apparatus. A heater may be provided as part of the system for temperature control and for heating and evaporating the wasted liquid.

In performing the analysis of collected drops by the apparatus of the present invention, the various optical, pressure, and electrical signals may be sent to a signal processing computer to perform the detection and analysis. Instructions may be sent out to control the fluid movement, temperature, optical intensity, and other parameters within the apparatus, or an alarm may be sounded if a hazardous contaminant is detected. In a further embodiment, a computer network may be provided to efficiently communicate with central locations for signal analysis, decision making, or alarming.

### BRIEF DESCRIPTION OF THE DRAWINGS

The invention will be described by reference to the following drawings, in which like numerals refer to like elements, and in which:

FIGS. 1A-1E are cross-sectional illustrations of liquid drops prior to discharge from channel outlets of the applicants' apparatus, such outlets having varying geometry;

FIG. 2 is a cross-sectional illustration of a single channel collection and detection apparatus of the present invention comprising droplet-stabilizing adjacent surfaces;

FIG. 3 is a cross-sectional illustration of a multi-channel collection and detection apparatus of the present invention, comprised of a plurality of the channels depicted in FIG. 2;

FIG. 4 is a cross-sectional illustration of a liquid drop formed within a channel and surfaces of the apparatus of FIG. 2, with the apparatus further comprising optical detection means;

FIG. 5 is a top view of another embodiment of a multi-channel collection and detection apparatus of the present invention;

FIG. 6 is an exploded perspective view of another multi-channel collection and detection apparatus comprising a two dimensional array of channels;

FIG. 7 is a cross-sectional view of one embodiment of a cylindrical collection and detection apparatus comprising a first annulus;

FIG. 8 is a cross-sectional view of a multi-channel collection and detection apparatus comprising an inner annulus and an outer annulus;

FIG. 9 is a cross-sectional illustration of another embodiment of the applicants' apparatus, comprising a hydrophilic surface surrounding the optical path in the solid therein, by which contaminant containing moisture may be condensed within such optical path and analyzed optically;

FIG. 10 is a simplified perspective view of a multi-droplet cartridge-type lo embodiment of the device of FIG. 9;

FIG. 11 is a top cross-sectional view of the embodiment of FIG. 10 taken along the line 11-11 of FIG. 10;

FIGS. 12A-12C are cross-sectional views of a single channel collection and detection apparatus of the present invention comprising a porous medium for subsequent collection of contaminant containing liquid collected by a drop in a flowing air stream;

FIGS. 13A-13C are cross-sectional views of a single channel collection and detection apparatus of the present invention comprising an indicating reservoir for reaction with and indication of the contents of contaminant containing liquid collected by a drop in a flowing air stream;

FIG. 14 is a cross-sectional view of a collection and detection apparatus similar to the apparatus of FIGS. 13A-13C, but further comprising a liquid reservoir to collect sampled liquid therefrom;

FIGS. 15A-15C are cross-sectional sequential illustrations of the bridging of a gap by a liquid drop within a detection channel in an embodiment of the applicants' apparatus further comprising hydrophilic surfaces that include reactive species;

FIG. 16 is a cross-sectional illustration of a further embodiment of the applicant's apparatus wherein light transmitted through a droplet is focused by the meniscus of such drop within a detection chamber;

FIG. 17 is a cross-sectional illustration of a further embodiment of the applicant's apparatus wherein light is reflected from the meniscus of a drop to an optical sensor within a detection chamber;

FIG. 18 is a simplified perspective view of a multi-drop embodiment of the collection and detection apparatus of FIG. 17;

FIGS. 19A and 19B are side and top views, respectively, of means to detect the size of a drop by detecting electrical resistance change when such drop wets a substrate coated with conductive material;

FIG. 20A is a side cross-sectional view of another multi-channel collection and detection apparatus of the present invention;

FIG. 20B is a of the apparatus of FIG. 20A taken along the line 20B-20B of FIG. 20A; and

FIG. 21 is a perspective sectional view of a cylindrical embodiment of the multi-channel collection and detection apparatus of FIGS. 20A and 20B.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by the appended claims.

### DESCRIPTION OF THE PREFERRED EMBODIMENTS

For a general understanding of the present invention, reference is made to the drawings. In the drawings, like reference numerals have been used throughout to designate identical elements.

As used herein, the term “hazardous contaminant” is meant to indicate a substance that has an adverse effect on another entity in the environment in which the substance is present. In particular, the adverse effect may be direct short-term or long term harm to the health of a human or other life form. The adverse effect may be the pollution of another material used by the human or other life form, e.g. drinking water. The adverse effect on a non-living entity may be a degradative effect, for example, the corrosion of a metal. In this specification, the terms “hazardous contaminant” and “contaminant” are used interchangeably, with the latter being used for the sake of brevity.

It is to be understood that in the description of the present invention in this specification and drawings, the present invention is described in the context of collecting and detecting materials that are considered “hazardous contaminants.” This is by no means meant to be taken as limiting the scope of the present invention to the detection solely of hazardous substances. It is to be understood that the apparatus and methods of the present invention may be used for real time detection of the presence of substances for which such presence is desirable. In general, the present invention is broadly directed to low cost, sensitive, continuous and automatic methods and apparatus for the collection and detection of a wide range of substances contained in a gas, and in particular, ambient air, including but not limited to gaseous, solid, liquid, aerosol, organic, inorganic, hydrophobic, hydrophilic, radioactive, living, and non-living substances. Such living substances may include microbes such as e.g., bacteria, spores, viruses, and the like.

Some of such substances may be gaseous atomic species within the flowing gas or flowing air stream, e.g. an asphixiant such as a nobel gas; or such substances may be a gaseous molecular species such as phosgene (COCL₂); or such substances may be entrained particulate substances including but not limited to aerosols, dusts, and microbes. In the latter case, such particulates that are detectable by the apparatus and methods of the present invention generally have characteristic dimension (such as diameter if substantially spherical) on the order of between about 0.1 nanometers to about 100 micrometers.

In certain situations where hazardous airborne hazardous contaminants may be present, the concentration of the airborne hazardous contaminants may be very low. Such low concentration may be lower than the detection capabilities of conventional analytical devices, particularly devices that are compact, portable, simple to use, inexpensive, and that provide rapid delivery of analytical results. In the present invention, a streaming sample of air containing airborne hazardous contaminants is directed over a liquid surface, or a plurality of surfaces, such that some of the hazardous contaminants are captured at the liquid surface and/or transferred into the bulk liquid. In the preferred embodiment, such liquid surface or surfaces are air-liquid interfaces. Once transferred into the bulk liquid or suspended on the air-liquid interface, such hazardous contaminants are detectable by the apparatus and methods of the present invention.

In the applicants' apparatus, the sensitivity of detection is dependent upon the concentration of the contaminants transferred into the liquid. In order to increase the sensitivity of airborne contaminant detection in the applicant's apparatus, the ratio between the surface area and the volume of the liquid that collects the airborne contaminants is made large. In the present invention, the collection liquid is provided in the form of small liquid drops. A small liquid drop has a very large surface area to volume ratio, which simultaneously enhances the efficiency of contaminant capture and creates a high concentration of collected contaminant species in the liquid.

It is known that most airborne contaminants have great affinity to water or other liquids. Such contaminants dissolve or suspend in water or other liquids upon contact. In one embodiment of the present invention, tiny liquid drops are used to continuously collect airborne contaminants from ambient air. Additionally, in order to increase the efficiency of capturing charged airborne contaminants, positive electric charge may be applied to the liquid drop(s) to facilitate capturing negatively charged contaminants, and negative charge may be applied to the liquid drop(s) to facilitate capturing positively charged contaminants. In a further embodiment, lowering the temperature of the collection liquid also enhances contaminant capture efficiency due to reduced kinetic energy of such contaminants. In a further embodiment, chemicals provided in the liquid can react with captured contaminants. Such a reaction depletes captured contaminants in the liquid, which will enhance capture efficiency, especially for contaminants a with very low concentration in the air.

The collection liquid drops may be generated by at least three different methods. Each of these methods has in common the feature wherein a gas-liquid interface is provided, and such gas-liquid interface is made sufficiently small so as to be stabilized and maintained in a substantially constant position by surface tension. In one preferred method, liquid drops are created by driving the liquid flowing out of a small channel, such channel preferably comprising an outlet having a diameter less than about 2 millimeters. The surface surrounding the channel outlet is preferably hydrophobic to prevent the spreading of the liquid when a drop exits from the channel outlet and breaks with the flowing bulk liquid stream. To maintain the stability of the liquid drop, the volume of the liquid drop is preferably smaller than about 100 micro-liters.

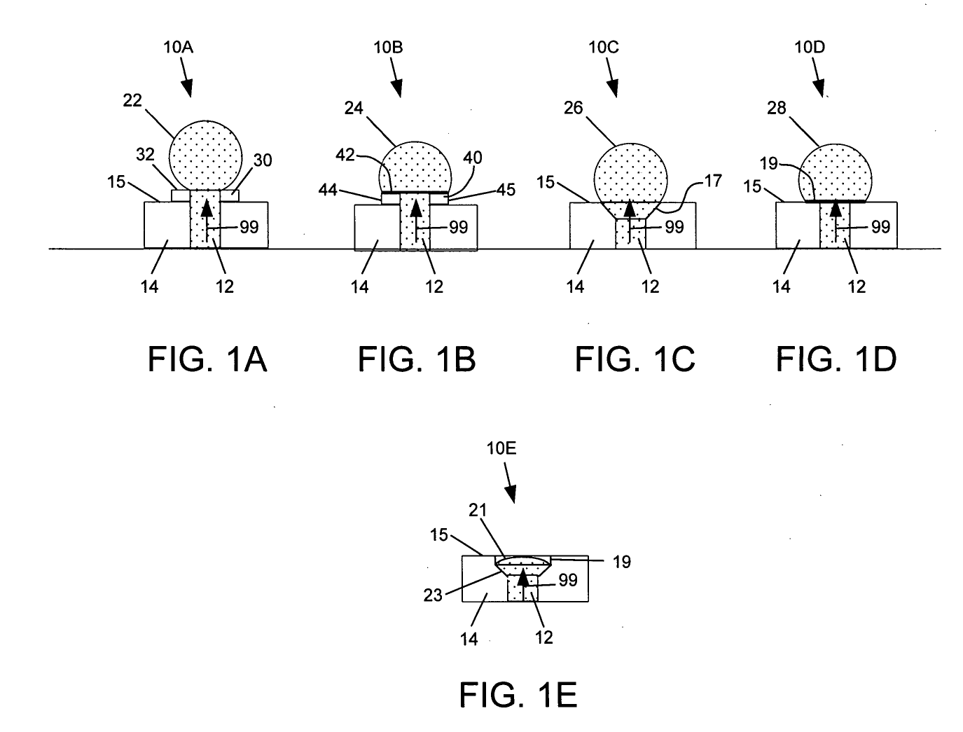

The liquid drop size and stability can be controlled by the geometry and surface wettability of the channel outlet. FIGS. 1A-1E are cross-sectional illustrations of liquid drops prior to discharge from channel outlets of the applicants' apparatus, such outlets having varying geometry. For each of droplet generators 10A, 10B, 10C, 10D, and 10E, droplets are generated by delivering collection liquid through channel 12 formed in wall 14, as indicated by arrow 99. In general, such droplets are stabilized by surface tension and by the hydrophobicity of the solid surface upon which they are in contact, if the liquid droplets are hydrophilic. The inside wall of the channels are preferably wettable to the liquid used although a non-wettable channel may also function satisfactorily.

Referring in particular to FIG. 1A, liquid drop 22 has been formed and is disposed upon on a hydrophobic step 30, which in turn is joined to hydrophobic surface 15 of wall 14. Top surface 32 of step 30 is hydrophobic. Referring to Figure 1B, liquid drop 24 is disposed upon a hydrophilic step 40, which is comprised of hydrophilic top surface 42 and hydrophobic sides 44 and 45. Referring to FIG. 1C, liquid drop 26 is disposed upon hydrophobic surface 15 of wall 14. Channel 12 is comprised of an expanding tapered outlet 17 in hydrophobic surface 15, dimensioned such that liquid drop 26 is partially contained in tapered outlet 17. It is not required that liquid drop 26 extend to as complete a spherical shape as is depicted in FIG. 1C, although such extension provides a greater surface area to volume ratio. The free liquid surface may only extend up into the tapered outlet 17 to a level approximately that of surface 15, and still provide some functionality in the present invention.

Referring to FIG. 1D, liquid drop 28 is disposed upon a hydrophilic area 19, which is surrounded by hydrophobic surface 15. In FIGS. 1A-1D, all of the channels 12 are hydrophilic. The configurations of FIGS. 1B-1D have the flexibility of controlling liquid drop size by changing the surface geometry without affecting the liquid channel.

Referring to FIG. 1E, liquid drop 21 is disposed upon a sharp non-wettable (hydrophobic) step 19 in solid 14 under the surface 15. This step stabilizes the liquid drop inside the expanding tapered hydrophilic outlet 23.

Examples of suitable materials for the hydrophobic surfaces of the apparatus of the present invention include but are not limited to fluoropolymers such as e.g., poly-tetrafluoroethylene (PTFE), also known as Teflon®. Numerous other fluoropolymers and other hydrophobic plastics such as polypropylene, and polyethylene, that are easily molded or otherwise formed will be apparent to those skilled in the art. The surface of the materials used for the apparatus of the present invention can also be modified by coating specific materials (such as Polysiloxanes) to make such surfaces hydrophobic.

In the preferred embodiment, the diameter of channel 12 of FIGS. 1A-1D is preferably less than 2 mm, although devices with larger diameters may still able to generate stable liquid drops depending upon the rheology and surface tension of the particular fluid. The largest diameter of the liquid drop 22-28 is preferably less than about 10 mm.

The aforementioned drop generator configurations are intended to be illustrative and not limiting. It will be apparent to those skilled in the art that numerous other drop generation methods may be suitable. For example, a needle may be used to generate and hold a liquid drop. An optical fiber may hold a liquid drop at its distal end and with the light passing through the optical fiber from the proximal end to the distal end and through the liquid drop. In one embodiment, an optical sensor may be used to detect the light passing through the liquid drop. In addition, the shape of the liquid drop(s) in the apparatus of the present invention do not have to be of a perfectly spherical shape. Such a drop may have an arbitrary shape as long as the drop is stabilized by surface energy at the air-liquid solid interface, i.e. the contact line of the drop with the solid.

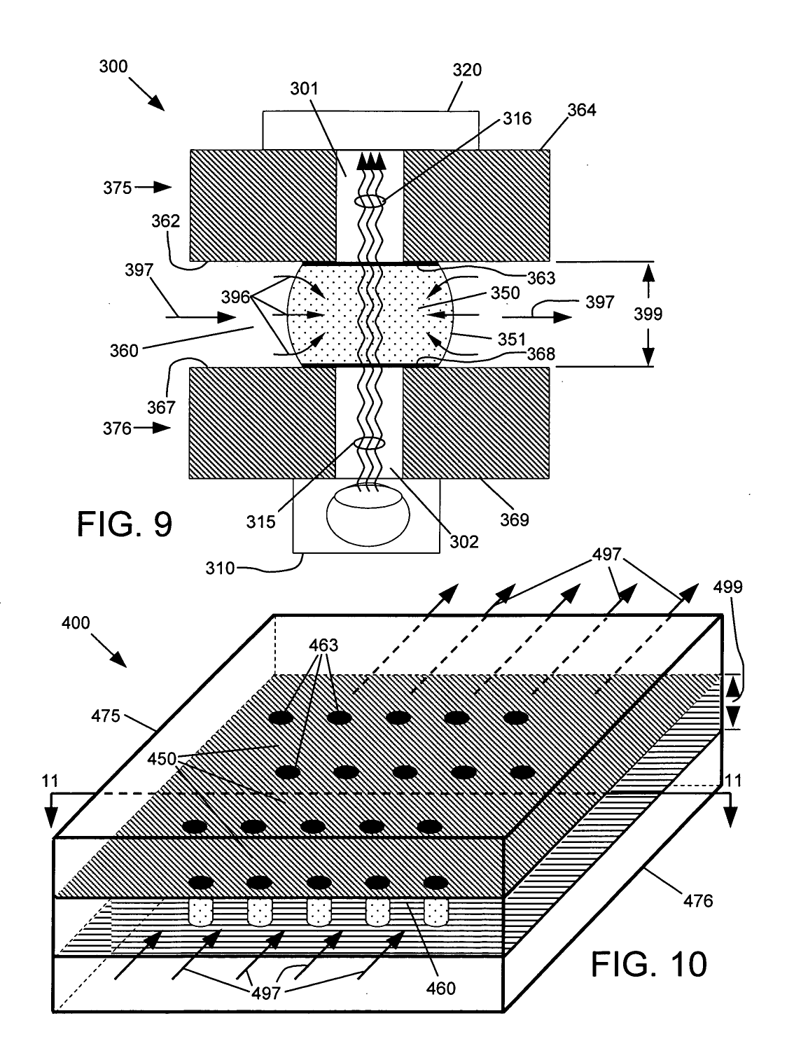

A second method to generate a liquid water drop on a surface comprises the steps of providing a solid surface with special patterns of hydrophobicity and hydrophilicity, cooling such solid surface to a temperature less than the dew point of the water vapor in the ambient air, and condensing liquid water from the air. Such liquid phase will condense preferentially to the hydrophilic area when its temperature is lower than the dew point at the solid surface. Condensed liquid micro-droplets will coalesce and grow to form larger liquid drops over time. In the interim, airborne contaminants will be captured by the condensed liquid. As was described previously, the stability of such liquid drops may be maintained by the geometry and wettability of the solid surface, as well as the surface tension of the liquid. The hydrophilic area of such a solid surface is preferably less than 1 mm². One embodiment of this method is depicted in FIGS. 9-10, and will be described subsequently in this specification.

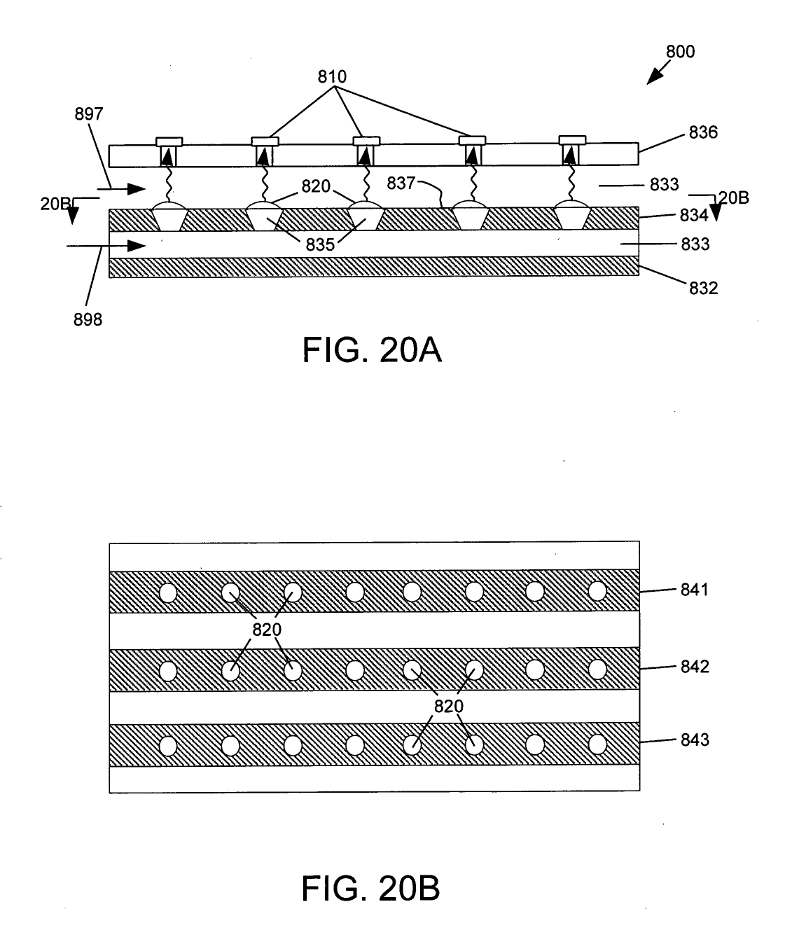

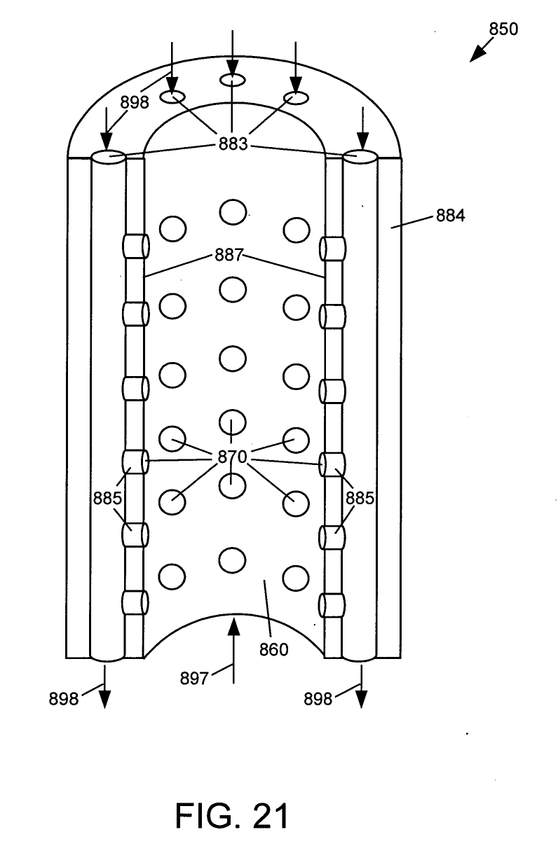

A third method depicted in FIGS. 20A-21 and described subsequently herein to generate liquid drops is to pass liquid through a channel comprising small holes along the wall thereof. These holes allow the liquid inside the channel to be exposed to the ambient air external to the channel. The inside of the channel and the hole through the channel wall are hydrophilic so that liquid can flow easily without generating bubbles when liquid is first applied to the system. The outer surface of the solid channel wall is hydrophobic so that the liquid drop will not spread out across the solid surface when it exits the hole of the channel. The edge of the hole is provided with a sharp transition so that the meniscus may be limited at the hole and exposed to the air instead of spreading out across the surface of the solid. With this construct, the meniscus can be withdrawn to inside the channel by a pump for further analysis or liquid collection or disposal.

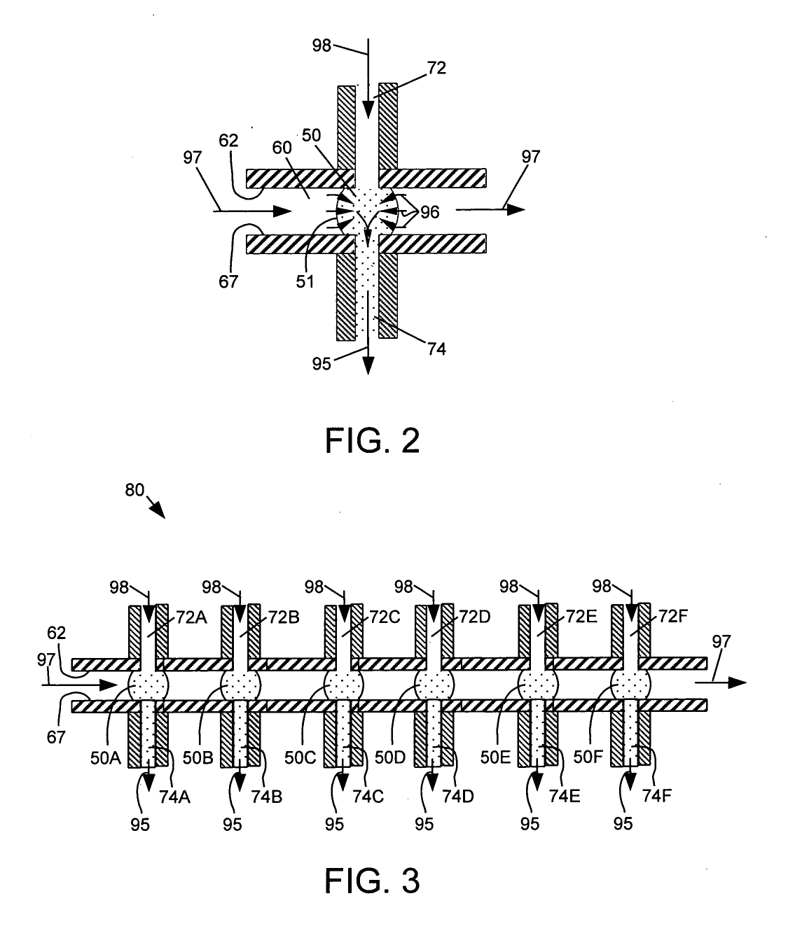

In one preferred embodiment, two solid adjacent surfaces are used to further stabilize the liquid drop formed at the outlet of a channel, and to provide a chamber for the stream of contaminant-containing air to be analyzed by the applicants' device. It is preferable that such adjacent surfaces are parallel, although some minor deviation therefrom may occur and still provide a suitable chamber for use in the present invention. FIG. 2 is a cross-sectional illustration of a single channel collection and detection apparatus of the present invention comprising droplet-stabilizing adjacent surfaces. Referring to FIG. 2, a liquid drop 50 between two solid hydrophobic surfaces is created by the capillary flow between surfaces 62 and 67 of chamber 60. Such capillary flow forms meniscus 51, which provides a surface area for capture and transfer of contaminants in the flowing air stream in chamber 60 from such air stream into flowing collection liquid. Collection liquid enters chamber 60 through longitudinal inlet channel 72 as indicated by arrow 98. Contaminant-containing air flowing within chamber 60 as indicated by arrows 97 impacts liquid drop 50, and contaminants are captured at meniscus 51 and transferred into the bulk volume of droplet 50 as indicated by arrows 96. Captured contaminants are subsequently entrained in the flowing capture liquid, and discharged out of chamber 60 through longitudinal outlet channel 74, as indicated by arrow 95. The captured contaminants within the exit stream are carried to a downstream location (not shown) for analysis. The surfaces 62 and 67 are non-wettable to the contaminant-collecting liquid, and the liquid drop 50 is stabilized by the surface tension. The size of liquid drop 50 is controlled primarily by the flow rate that is maintained at the inlet and outlet of the two capillaries or channels 72 and 74 connecting the liquid drop, said channels 72 and 74 being coaxially aligned with each other along their longitudinal axes, i.e. in the direction of arrows 98 and 95.

Without wishing to be bound to any particular theory, applicants believe that contaminants are captured in a proportionately greater amount on the upstream side of drop 50, but that some capture may occur along the remainder of the perimeter of drop 50. Applicants further believe that transfer of contaminants from the surface of drop 50 to the bulk liquid may be enhanced by flow instabilities which occur within drop 50, and by possible oscillations of the position of the contact lines between drop 50 and surfaces 62 and 67. (It is to be understood that such contact “lines” are actually circles of contact between drop 50 and surfaces 62 and 67.)

FIG. 3 is a cross-sectional illustration of a multi-channel collection and detection apparatus of the present invention, comprised of a plurality of the channels depicted in FIG. 2. The apparatus of FIG. 3 provides the capability to perform multiple analyses simultaneously, multiple liquid drops between a pair of parallel solid surfaces are created, and multiple individual streams of captured contaminants are delivered through multiple individual channel for analysis with different assays or optical detection. Referring to FIG. 3, channel array 80 is comprised of adjacent surfaces 62 and 67 forming chamber 60, through which flows contaminant-containing air stream as indicated by arrows 97. Collection liquid enters chamber 60 through a plurality of inlet channels 72A-72F as indicated by arrows 98. Contaminant-containing air flowing within channel 60 as indicated by arrows 97 impacts liquid drops 50A-50F, and contaminants are captured at the menisci thereof, transferred into their respective bulk volumes, subsequently entrained in the flowing capture liquid, and discharged out of chamber 60 through outlet channels 74A-74F, as indicated by arrows 95. As indicated previously, these multiple individual streams of captured contaminants from channels 74A-74F are delivered to various apparatus (not shown) for analysis with different assays or optical detection. It will be apparent that channel array 80 may be provided with less or more droplet-forming channels, depending upon the situation and the number of individual or redundant analyses to be performed.

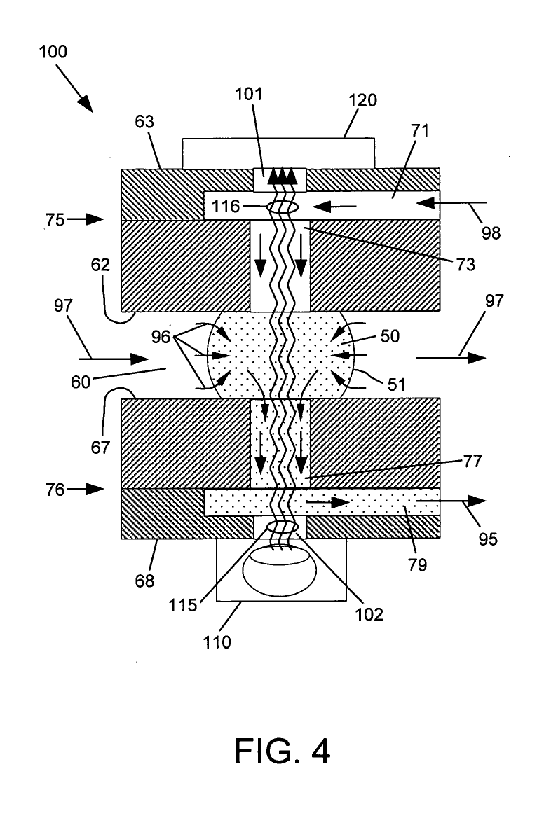

In a further embodiment of the applicants' apparatus, contaminant detection means are integrated into the contaminant-capturing droplet formation device. FIG. 4 is a cross-sectional illustration of a liquid drop formed within a channel and surfaces of the apparatus of FIG. 2, with the apparatus further comprising optical detection means. Referring to FIG. 4, there is depicted a single channel collection and detection device of the present invention comprising droplet-stabilizing adjacent surfaces similar to the device of FIG. 2 previously described herein. Device 100 comprises bulk adjacent walls 75 and 76 having hydrophobic surfaces 62 and 67, between which flows a contaminant-containing air stream as indicated by arrows 97. A droplet 50 is formed between surfaces 62 and 67, wherein such droplet captures contaminants from the air stream as indicated by arrows 96.

Bulk wall 75 is provided with a first entry channel 71 connected to and approximately perpendicular to a second entry channel 73 for the entry of contaminant capturing liquid as indicated by arrow 98. Bulk wall 76 is provided with a first exit channel 77 connected to and approximately perpendicular to a second entry channel 79 for the exit of liquid containing captured contaminants as indicated by arrow 95. Bulk wall 75 is further provided with an optically transparent window 101, and bulk wall 76 is further provided with an optically transparent window 102. In this manner, there is provided an optical pathway through bulk wall 75, through the flowing contaminant-capturing liquid within channels 71 and 73, through the contaminant-containing liquid within droplet 50, channel 77, and channel 79, and through bulk wall 76.

This optical pathway is used in the present invention, wherein optical detection means are provided integrally within device 100 for the detection of contaminants in the flowing air stream, after such contaminants have been captured by drop 50. Referring again to FIG. 4, optical detection means comprises light source 110 disposed upon the exterior surface 68 of wall 76, and optical sensor 120 disposed upon exterior surface 63 of wall 75. In use, light rays 115 emanate from light source 110, passing through the contaminant-containing liquid stream to produce attenuated rays 116, which are detected by optical sensor 120. Attenuated rays 116 thus provide a continuous real-time indication of the presence and quantity of contaminants within droplet 50 and the liquid stream in channels 77 and 79, and thus contaminants within the air stream flowing through chamber 60.

Attenuated rays 116 may be analyzed to determine optical absorbance of the liquid within the optical path at a certain wavelength and/or attenuated rays 116 may be analyzed by other known optical spectral analysis methods. For example, organic disulfide compounds are known to have an absorbance maximum at about 194 nanometers of wavelength. Hence an optical spectroscopic analysis method using ultraviolet light of this wavelength may be used to detect such compounds.

In one preferred embodiment, light source 110 provides substantially the entire available spectrum from the infrared spectrum of about 1000 micrometers in wavelength to about 700 nanometers in wavelength; through the visible spectrum of about 700 to about 400 nanometer in wavelength; to the ultraviolet spectrum of about 400 nanometer to about 150 nanometers in wavelength. This is preferable in order for the apparatus 100 to detect many different types of airborne contaminants and to detect changes in coloration of the droplet due to the presence of contaminants, or the presence of colored chemical indicators that generate a color change upon exposure to certain contaminants, as will be described subsequently in this specification.

In the embodiment depicted in FIG. 4, optical light source 110 is depicted as an integrated device, with connections providing electrical energy not shown. It will be apparent that in alternative embodiments not shown, optical light source 110 may comprise an optical fiber connected to surface 68 of wall 76, such that light therefrom is directed through the optical path to optical sensor 120. In this manner, the light from multiple light sources (not shown), such as incandescent bulbs, ultra bright broad spectrum LED's, infrared LED's visible LED's, and/or various lasers may be combined and delivered through such optical fiber to the device 100. It will be further apparent that the arrangement of the optical light source 110 and the optical sensor 120 may be inverted with respect to walls 75 and 76, and still achieve the same result in the detection of contaminants. It will be further apparent that window 101 in wall 75 may be replaced with a mirror, and that optical light source 110 and optical sensor 120 may be combined in place of optical light source 110 of FIG. 4, thus providing the same capabilities in device 100.

In device 100 depicted in FIG. 4, the size and shape of the liquid drop may be controlled such that the free surface (meniscus) of the liquid drop does not interfere with the optical path. Either an optical method and/or a pressure method may be used to monitor the size of the liquid drop. In one embodiment employing an optical method, if the liquid drop 50 is too small, the free surface 51 will interfere with the optical sensor signal. That interference can be detected as a signal to increase the size of liquid drop 50. In another embodiment employing a pressure method, a small passageway is provided that connects the drop at surface 62 of surface 67 to a pressure sensor, in order to measure the internal pressure of the drop 50. If pressure generated by the surface tension of the liquid drop 50 is not within a specified range (i.e. too small or too large), the size of liquid drop 50 is then determined to be either too small or too large and is adjusted accordingly.

The preceding description is illustrative of a single-channel contaminant collection and detection device. In a further embodiment, there is provided a multi-channel collection and detection apparatus comprising a linear array of channels such as is depicted in FIG. 3, each being provided with optical detection means as described herein. Such optical detection means may vary with respect to the particular light delivered to each channel: some channels may receive infrared light, some channels may receive visible light, and some channels may receive ultraviolet light. Some channels may receive multiple narrow bands of the spectrum at specific wavelengths. The choices of light employed will depend upon the particular contaminants to be detected, and their optical properties with respect to certain wavelengths.

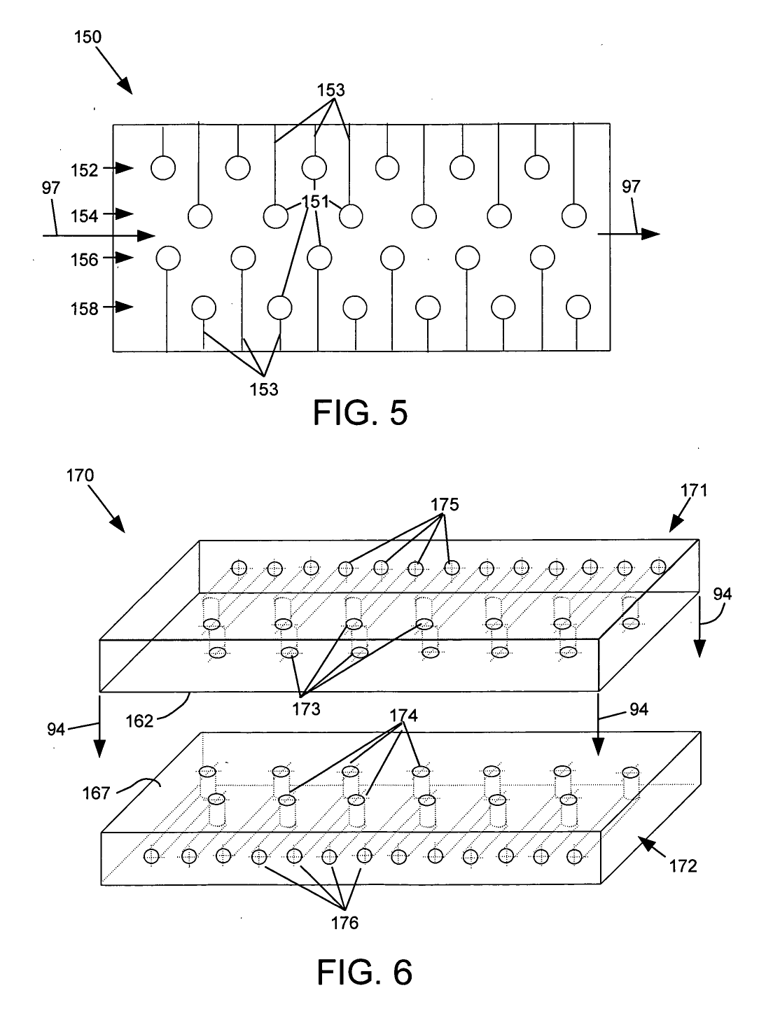

In a further embodiment, there is provided a multi-channel collection and detection apparatus comprising a two dimensional array of channels. FIG. 5 is a top view of such a multi-channel collection and detection apparatus, illustrated schematically. Referring to FIG. 5, channel array 150 comprises rows 152, 154, 156, and 158 of channels. Each of rows 152-158 comprise a linear array of channels as described previously herein. Each individual channel 151 is connected to an orthogonal delivery channel 153, and each individual channel 151 is provided with optical detection means as described previously herein and depicted in FIG. 4.

In the preferred embodiment, rows 152, 154, 156, and 158 are staggered with respect to each other as shown in FIG. 5. This arrangement provides for a more dense packing of channels within array 150, and a more beneficial air path through array 150. However, it will be apparent that channels 151 may be arrayed in other suitable configurations that will achieve the desired result.

FIG. 6 is a perspective exploded view of another multi-channel collection and detection apparatus comprising a two dimensional array of channels. Referring to FIG. 6, array 170 comprises a first half 171 and a second half 172. In use, first half 171 and second half 172 are moved toward each other as indicated by arrows 94, and are disposed in close proximity to each other, such that a gap is provided between surface 162 of half 171 and surface 167 of half 172. The gap between these surfaces forms the chamber (not shown) for the formation of droplets, as has been previously described herein for chamber 60 of the devices of FIGS. 2, 3, and 4.

Referring again to FIG. 6, when halves 171 and 172 are matched together to form a gap therebetween, the arrays of channels 173 and 174 are aligned to produce aligned channels between which droplets may be formed for the collection and analysis of contaminants captured from an air stream flowing between surfaces 162 and 167 as described previously herein. When array 170 is in use, contaminant capturing liquid is delivered into side entry ports 175 of half 171, and contaminant-containing liquid is delivered out of side entry ports 176 of half 172.

For each pair of matched channels 173 and 174, there is provided optical detection means as shown in FIG. 4 and previously described herein. In one preferred embodiment, halves 171 and 172 are made of optically clear materials such as polymethylmethacrylate plastic, polycarbonate plastic, glass, or quartz. In this manner, there is no need to provide optically clear windows in halves 171 and 172 as described previously herein.

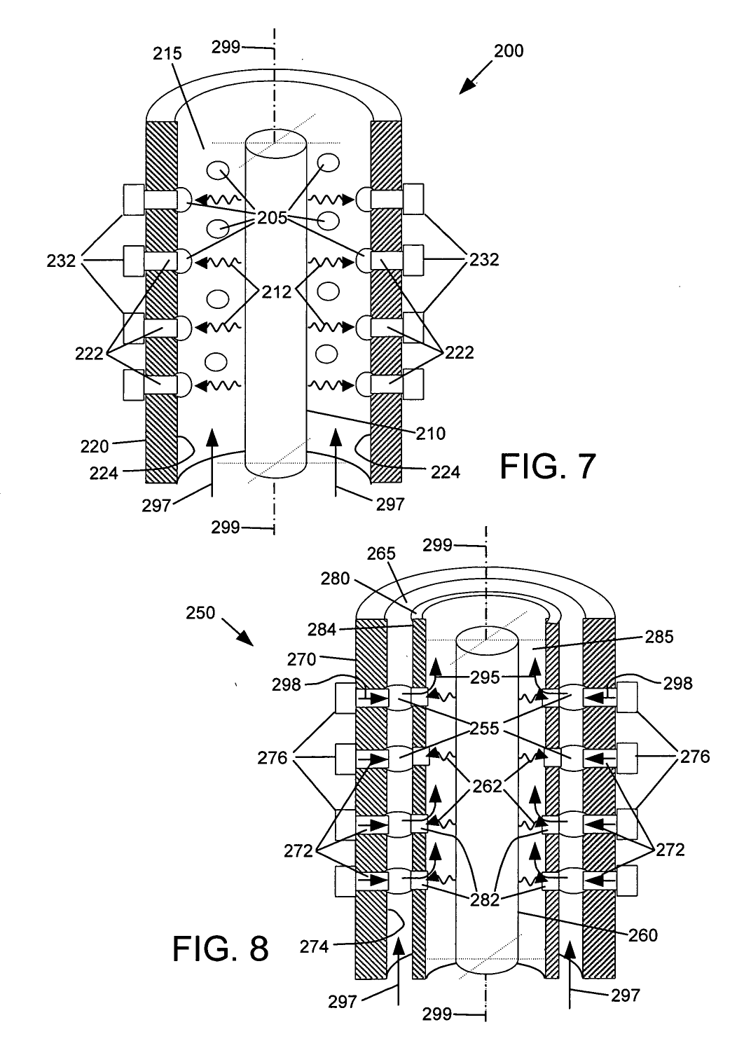

FIG. 7 is a cross-sectional view of one embodiment of a cylindrical collection and detection apparatus comprising a first annulus. Referring to FIG. 7, apparatus 200 comprises a cylindrical light source 210 disposed within cylindrical shell 220, said light source 210 emanating light radially outwardly as indicated by arrows 212. The central axis of light source 210 is preferably collinear with the central axis of cylindrical shell 220 as indicated by line 299. Cylindrical shell 220 is provided with a plurality of channels 222 disposed in the wall thereof.

In operation, a contaminant-containing air stream is delivered through the annulus 215 that is formed by cylindrical light source 210 and cylindrical shell 220, as indicated by arrows 297. The contaminant-containing air stream flows past droplets 205, which are formed at the outlets to channels 222, which are connected to liquid supply channels. For the sake of simplicity of illustration, such supply channels are not shown, but may be provided with substantially the same structure as is depicted in FIG. 4 for supply channel 71, which connects to channel 73.

Thus, as the contaminant-containing air stream is delivered past droplets 205, some portion of the contaminant(s) is captured by droplets 205, as has been described previously herein. The presence of such contaminant in droplets 205 attenuates the light 212 emanating from light source 210, which passes through droplets 205, on through the liquid in channels 222, and on to optical sensors 232. Optical sensors 232 are used to detect and analyze the contents of droplets 205, thus identifying and quantifying what contaminants are present in droplets 205, and in the air stream flowing in the annulus 215.

In the preferred embodiment, the inner wall 224 of cylindrical shell 220 consists essentially of hydrophobic material, such that if the contaminant capturing liquid is hydrophilic, such droplets 205 are stabilized at the respective outlets of channels 222, and spreading of droplets 205 along the inner wall 224 of shell 220 is prevented. Such hydrophobic material may be provided as a coating on the inside of cylindrical shell 220, or such materials may be provided as steps, as shown in FIGS. 1A-1B and described previously herein. It will be apparent to those skilled in the art that the device as shown in FIG. 7 is essentially the cylindrical analog of the structures of FIGS. 1A-1D having planar geometry. It is to be understood that the vertical orientation of apparatus 200 is not required for such apparatus to function, and that such apparatus may be oriented horizontally, or at some other angle between horizontal and vertical.

In one embodiment, cylindrical light source 210 a cylindrical lamp. In other embodiments, cylindrical light source 210 may comprise an optical assembly including optical fibers, and means to direct light radially outwardly, such as mirrors or other optical elements. Cylindrical light source 210 is preferably provided with the capability of delivering light of varying wavelengths.

FIG. 8 is a cross-sectional view of a multi-channel collection and detection apparatus comprising a first annulus and a second annulus. Referring to FIG. 8, apparatus 250 comprises a cylindrical light source 260 disposed within an inner cylindrical shell 280, said light source 260 emanating light radially outwardly as indicated by arrows 262. The central axis of light source 260 is preferably collinear with the central axis of cylindrical shell 280 as indicated by line 299. Inner cylindrical shell 280 and cylindrical light source 260 are further disposed within outer cylindrical shell 270, the central axis of which is also collinear with the central axis of inner shell 280 and light source 260 as indicated by arrow 299.

Inner cylindrical shell 280 is provided with a plurality of channels 282 disposed in the wall thereof, and outer cylindrical shell 270 is provided with a plurality of channels 272 disposed in the wall thereof, such that channels 272 and 282 are aligned with each other and substantially coaxial and orthogonal with respect to central axis 299.

In operation, a contaminant-containing air stream is delivered through the outer annulus 265 that is formed by cylindrical inner shell 280 and cylindrical outer shell 270, as indicated by arrows 297. The contaminant-containing air stream flows past droplets 255, which are formed at the outlets to channels 272, which are connected to liquid supply channels. For the sake of simplicity of illustration, such supply channels are not shown, but may be provided with substantially the same structure as is depicted in FIG. 4 for supply channel 71, which connects to channel 73. Droplets 255 are held between inner surface 274 of outer shell 270 and outer surface 284 of shell 280, in much the same manner as was described previously herein for droplets 50 of FIGS. 2-4.

Referring again to FIG. 8, as the contaminant-containing air stream is delivered past droplets 255, some portion of the contaminant(s) is captured by droplets 255, as has been described previously herein. The presence of such contaminant in droplets 255 attenuates the light 262 emanating from light source 260, which passes through droplets 265, on through the liquid in channels 272, and on to optical sensors 276. Optical sensors 276 are used to detect and analyze the contents of droplets 255, thus identifying and quantifying what contaminants are present in droplets 255, and in the air stream flowing in the annulus 265. Subsequent to the capture of contaminants, liquid from droplets 50 flows through channels 282 in cylindrical inner shell 280 into inner annulus 280 that is formed by cylindrical inner shell 280 and cylindrical light source 260 as indicated by arrows 295.

In the preferred embodiment, the inner wall 274 of cylindrical shell 270 and outer wall 284 of inner cylindrical shell 280 consist essentially of hydrophobic material, such that if the contaminant capturing liquid is hydrophilic, such droplets 255 are stabilized at the respective outlets of channels 272, and spreading of droplets 255 along the inner wall 274 of shell 270 and the outer wall 284 of inner cylindrical shell 280 is prevented. Such hydrophobic materials may be provided as a coating on surfaces 274 and 284, or such materials may be provided as steps, as shown in FIGS. 1A-1B and described previously herein. It will be apparent to those skilled in the art that the device as shown in FIG. 8 is essentially the cylindrical analog of the structures of FIGS. 2-4 having planar geometry. It is to be understood that the vertical orientation of apparatus 250 is not required for such apparatus to function, and that such apparatus may be oriented horizontally, or at some other angle between horizontal and vertical.

In one embodiment, cylindrical light source 260 a cylindrical lamp. In other embodiments, cylindrical light source 260 may comprise an optical assembly including optical fibers, and means to direct light radially outwardly, such as mirrors or other optical elements. Cylindrical light source 260 is preferably provided with the capability of delivering light of varying wavelengths.

FIG. 9 is a cross-sectional illustration of another embodiment of the applicants'apparatus, comprising a hydrophilic surface surrounding the optical path in the solid therein, by which contaminant containing moisture may be condensed within such optical path and analyzed optically as described previously herein. In this embodiment, no provision is made for the active delivery of liquid through the droplet disposed in the optical path. Referring to FIG. 9, device 300 comprises bulk adjacent walls 375 and 376 having hydrophobic surfaces 362 and 367, and hydrophilic dots 363 and 368 coated or otherwise formed on surfaces 362 and 367. Hydrophilic dots 363 and 368 are disposed opposite each other, and hydrophilic dots 363 and 368 are optically transparent, so as to allow light to pass through them. Hydrophobic surfaces 362 and 367, and hydrophilic dots 363 and 368 are separated from each other by a gap 399, which is preferably between about 0.5 and about 5 millimeters.

A contaminant-containing air stream is delivered between hydrophobic surfaces 362 and 367 and hydrophilic dots 363 and 368 as indicated by arrows 397. The air stream is highly saturated with water vapor, and hydrophobic surfaces 362 and 367 and hydrophilic dots 363 and 368 in particular are cooled below the dew point temperature of the water vapor in the flowing air stream. Hydrophilic dot surfaces 363 and 368 may be cooled by any suitable cooling means, such as coolant water jackets (not shown) circulating chilled coolant through cavities near such dots hydrophilic 363 and 368, or cooling fins on the outer surfaces of the respective walls located near such dots.

Hence the water vapor condenses preferentially onto hydrophilic dots, thereby growing individual drops on each dot until such drops become sufficiently large enough to touch each other. At this time, the drops combine into a single drop 350 that is disposed between hydrophilic dots 363 and 368 as depicted in FIG. 9. Alternatively, the flowing gas stream is provided with an increased vapor content such that the dew point temperature of the flowing gas stream is above the temperature of the wall surfaces, and condensation of vapor occurs on the wall surfaces.

During this growth process of the pair of drops, and subsequent to coalescence into drop 350, contaminants from the air stream are being captured at the surface thereof as indicated by arrows 396. Bulk wall 375 is provided with an optically transparent window 301, and bulk wall 376 is further provided with an optically transparent window 302. In this manner, there is provided an optical pathway through bulk wall 375, through the coalesced contaminant-capturing drop 350 between hydrophilic dots 363 and 368, and through bulk wall 376.

This optical pathway is used in the present invention much as was described previously herein for the embodiment of FIG. 4, wherein optical detection means are provided integrally within device 300 for the detection of contaminants in the flowing air stream, after such contaminants have been captured by drop 350. Referring again to FIG. 9, optical detection means comprises light source 310 disposed upon the exterior surface 369 of wall 376, and optical sensor 320 disposed upon exterior surface 364 of wall 375. In use, light rays 315 emanate from light source 310, passing through the contaminant-containing liquid drop 350 to produce attenuated rays 316, which are detected by optical sensor 320. Attenuated rays 316 thus provide a continuous real-time indication of the presence and quantity of contaminants within droplet 350, and thus contaminants within the air stream flowing through chamber 360.

If it is desired to operate apparatus 300 intermittently, or to sample multiple air streams, the rate of flow of the air stream entering chamber 360 may be increased to a sufficiently high velocity, such that drop 350 and any residue thereof is completely swept away from the surfaces of hydrophilic dots 363 and 368. For further cleaning, a brief burst of rinse water may be delivered through chamber 360. Subsequently the analysis cycle may be restarted, with a restored flow of the air stream as indicated by arrows 397; such air stream may be from the same source (not shown) or a different source (not shown).

It will be apparent that the apparatus of FIG. 9 may be used to condense organic chemical (hydrophobic) vapors from an airstream and detect contaminants therein; in such circumstances, the major portion of the surface of such an apparatus will be made hydrophilic, and the condensation seed dot surfaces will be made hydrophobic.

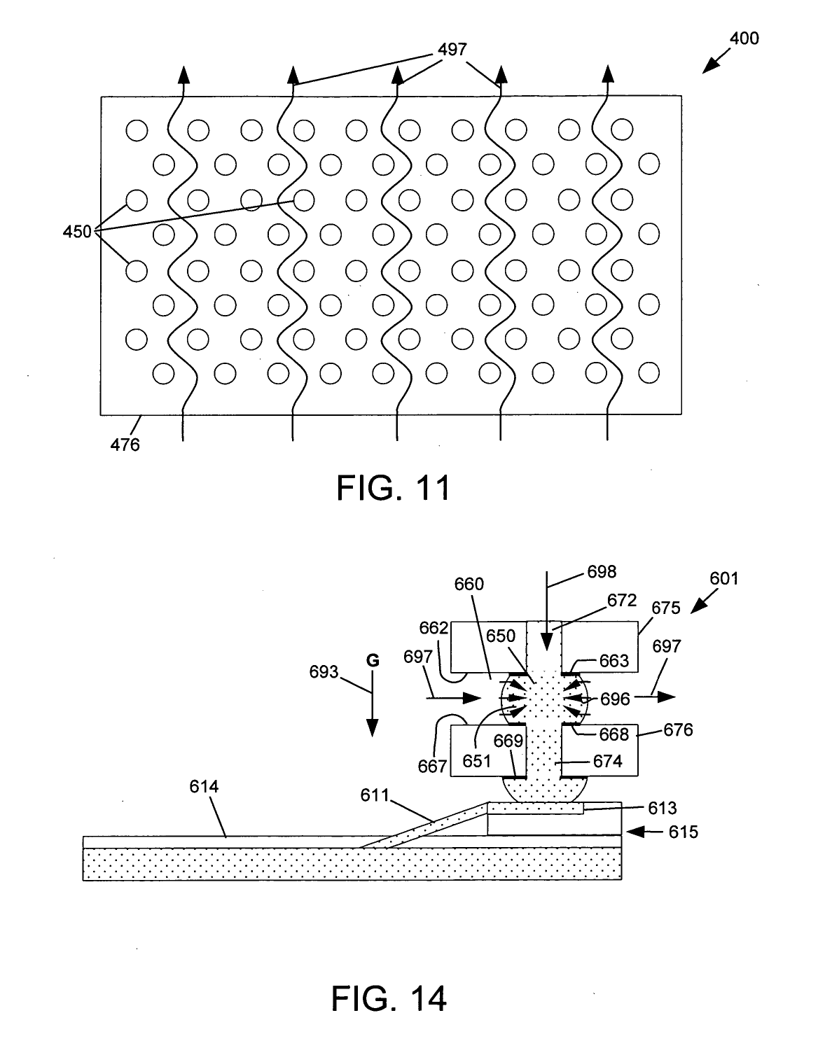

FIG. 10 is a simplified perspective view of a multi-droplet cartridge-type embodiment of the device of FIG. 9; and FIG. 11 is a top cross-sectional view of the embodiment of FIG. 10 taken along the line 11-11 of FIG. 10. Referring to FIG. 10 in particular, device 400 is comprised of upper wall 475 and lower wall 476, which form chamber 460 therebetween, having a gap height 499 of about the same dimensions as gap 399 of device 300 of FIG. 9. For the sake of simplicity of illustration, optical detection means as was shown and described in FIG. 9 are not shown in FIG. 10, but are provided for each of the drops 450 that condense on hydrophilic dots 463. Also for the sake of simplicity of illustration, drops 450 are depicted as being of a simple cylindrical shape, with it being understood that such droplets will have a shape as depicted for drop 350 of FIG. 9.

Referring also to FIG. 11, contaminant-containing air stream flows through chamber 460 as indicated by arrows 497. Drops 450 are formed by condensation onto hydrophilic dots 463 of wall 475, and correspondingly aligned hydrophilic dots (not shown) on wall 476, by lowering the temperature of walls 475 and 476 below the dew point of the air stream flowing therebetween. Rows of such dots are preferably arrayed in a staggered arrangement, such that the airflow follows a serpentine path as indicated in particular by arrows 497 in FIG. 11. Without wishing to be bound to any particular theory, applicant's believe that such a serpentine air flow path enhances the rate of transfer of contaminants into the droplets 450 in apparatus 400.

If it is desired to operate apparatus 400 intermittently, or to sample multiple air streams, the rate of flow of the air stream entering chamber 460 may be increased toga sufficiently high velocity, such that drops 450 and any residue thereof is completely swept away from the surfaces of the hydrophilic dots. For further cleaning, a brief burst of rinse water may be delivered through chamber 460. Subsequently the analysis cycle may be restarted, with a restored flow of the air stream as indicated by arrows 497; such air stream may be from the same source (not shown) or a different source (not shown). The optical detection means for detection and analysis of the various drops 450 may vary in order to analyze for different contaminants from the air stream, as was described previously herein for device 80 of FIG. 3.

In one embodiment (not shown) the optical detection means are made easily separable from a cartridge comprised of walls 475 and 476, and such cartridge is made disposable and is easily replaced with a new cartridge. In another embodiment (not shown), vibrational energy may be applied to the device to enhance the transport of the contaminant into the bulk of the liquid in each liquid drop 450. Such vibrational energy may be ultrasonic energy.

In another embodiment (not shown), the apparatus is not provided with means to lower the wall surface temperatures in order to cause condensation on the hydrophilic dots; instead, the walls are provided with droplets of contaminant capturing liquid already in place. Such droplets may be provided in small recesses on the wall surface, and may be covered over with a sealing film that prevents evaporation, and that is removed immediately prior to use.

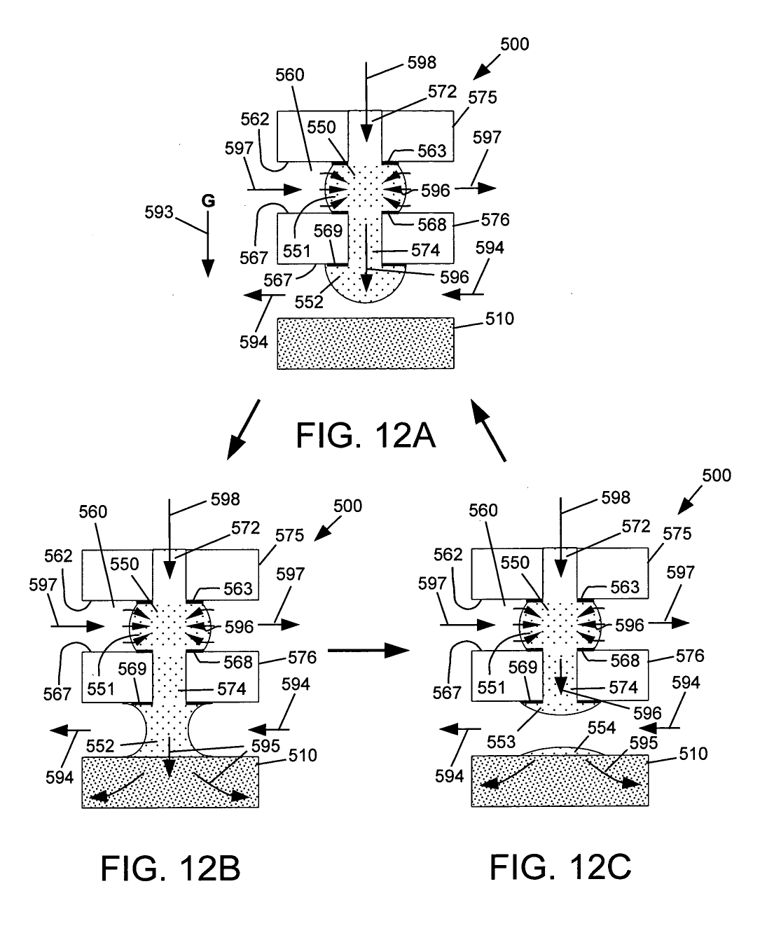

FIGS. 12A-12C are cross-sectional views of a single channel collection and detection apparatus of the present invention comprising a porous medium for subsequent collection of contaminant containing liquid collected by a drop in a flowing air stream. Referring to FIGS. 12A-12C, apparatus 500 comprises an upper wall 575 comprised of a hydrophobic surface 562 and a hydrophilic dot 563 surrounding the outlet of channel 572 into chamber 560; and a lower wall 576 comprised of a hydrophobic surface 567 and a hydrophilic dot 568 surrounding the inlet of channel 574 channel from chamber 560. During operation of apparatus 500, a liquid drop 550 between hydrophilic dots 563 and 568 is created by the capillary flow therebetween. Such capillary flow forms meniscus 551. Collection liquid enters chamber 560 through inlet channel 572 as indicated by arrow 598. Contaminant-containing air flowing within channel 560 as indicated by arrows 597 impacts liquid drop 550, and contaminants are captured at meniscus 551 and transferred into the bulk volume of droplet 550 as indicated by arrows 596.

Referring in particular to FIG. 12A, captured contaminants are subsequently entrained in the flowing capture liquid, and discharged out of chamber 560 through outlet channel 574, as indicated by arrow 596. Drop 552 forms on another hydrophilic dot 569, which is disposed on lower hydrophobic surface 567 of wall 576.

Drop 552 first forms as a sessile drop as depicted in FIG. 12A, since the apparatus is oriented as shown with respect to the force of gravity (G) as indicated by arrow 593. Referring to FIG. 12B, subsequently, drop 552 contacts hydrophilic porous medium 510, and is wicked away into such porous medium by capillary action therein, as indicated by arrows 595. Referring to FIG. 12C, when such contaminant-containing liquid has been sufficiently wicked away, drop 552 breaks into residual drop 553 on hydrophilic dot 569, and residual drop 554 on porous medium 510 that continues to be wicked away as indicated by arrow 595. The cycle of droplet formation and wicking into porous medium 510 then begins again and recycles continuously in the sequence shown in FIGS. 12A-12B-12C.

During this cycle, a second air stream flows between wall 576 and porous medium 510 as indicated by arrows 594. This airflow serves to dry porous medium before another liquid drop 552 forms and contacts porous medium 510. In one preferred embodiment, reactive chemicals are dispersed within porous medium 510 to facilitate the detection of contaminants captured in the liquid drops by causing a color change upon reaction with captured contaminants.

Porous medium 510 may consist essentially of any suitable hydrophilic material that may be formed with a high degree of porosity. Such media are well known in the filtration arts, and include but are not limited to ceramics, sintered metals, metal oxides such as titanium dioxide, and salts such as barium sulfate.

It will be apparent that additional apparatus may be provided which are comprised of multiple channel versions of the device 500 of FIGS. 12A-12C, in much the same manner as has been described previously herein for other embodiments of the applicants'collection and detection devices.

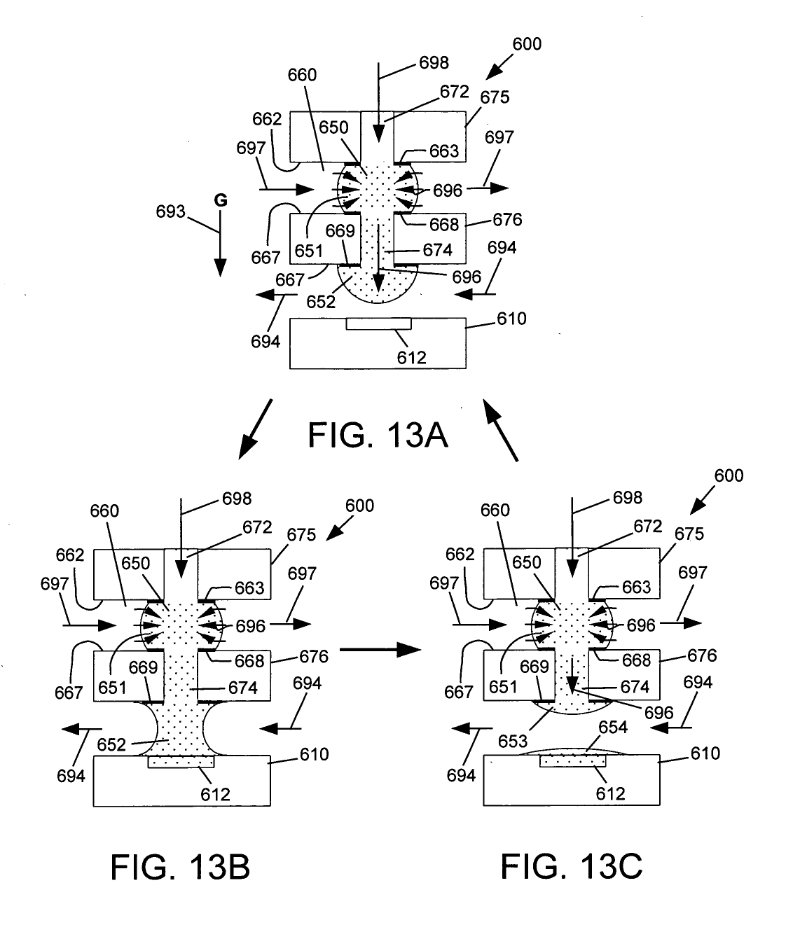

FIGS. 13A-13C are cross-sectional views of a single channel collection and detection apparatus of the present invention comprising an indicating reservoir for reaction with and indication of the contents of contaminant containing liquid collected by a drop in a flowing air stream. Referring to FIGS. 13A-13C, apparatus 600 comprises an upper wall 675 comprised of a hydrophobic surface 662 and a hydrophilic dot 663 surrounding the outlet of channel 672 into chamber 660; and a lower wall 676 comprised of a hydrophobic surface 667 and a hydrophilic dot 668 surrounding the inlet of channel 674 channel from chamber 660. During operation of apparatus 600, a liquid drop 650 between hydrophilic dots 663 and 668 is created by the capillary flow therebetween. Such capillary flow forms meniscus 651. Collection liquid enters chamber 660 through inlet channel 672 as indicated by arrow 698. Contaminant-containing air flowing within channel 660 as indicated by arrows 697 impacts liquid drop 650, and contaminants are captured at meniscus 651 and transferred into the bulk volume of droplet 650 as indicated by arrows 696.

Referring in particular to FIG. 13A, captured contaminants are subsequently entrained in the flowing capture liquid, and discharged out of chamber 660 through outlet channel 674, as indicated by arrow 696. Drop 652 forms on another hydrophilic dot 669, which is disposed on lower hydrophobic surface 667 of wall 676.

Drop 652 first forms as a sessile drop as depicted in FIG. 13A, since the apparatus is oriented as shown with respect to the force of gravity (G) as indicated by arrow 693. Referring to FIG. 13B, subsequently, drop 652 contacts indicating material 612 of bottom wall 610. Indicating material 612 contains at least one indicating chemical that undergoes a color change or another measurable change indicative of the occurrence of a reaction with a particular contaminant in drop 652. Such change may be detected, observed, recorded and/or transmitted to indicate that action should be taken in response to the presence of the contaminant.

Referring to FIG. 13C, when such contaminant-containing liquid has been sufficiently analyzed, and drop 652 has broken into residual drop 653 on hydrophilic dot 669, and residual drop 654 on bottom wall 610, the remaining portion of residual drops 653 and 654 may be swept away by a brief increase in the air stream indicated by arrows 694. The cycle of droplet formation and contact with indicating material 612 then begins again and recycles continuously in the sequence shown in FIGS. 13A-13B-13C.

It will be apparent that additional apparatus may be provided which are comprised of multiple channel versions of the device 600 of FIGS. 13A-13C, in much the same manner as has been described previously herein for other embodiments of the applicants'collection and detection devices.

FIG. 14 is a cross-sectional view of a collection and detection apparatus similar to the apparatus of FIGS. 13A-13C, but further comprising a liquid reservoir to collect sampled liquid therefrom. Referring to FIG. 14, apparatus 601 is comprised of substantially the same structure of walls 675 and 676, channels 672 and 674, and chamber 660 as apparatus 600 of FIGS. 13A-13C. Instead of a simple bottom wall 610 comprising an indicating material 612 (see FIG. 13A), apparatus 601 of FIG. 14 is further comprised of a reservoir assembly 615 comprising hydrophilic channel 611, which drains channel 613 into liquid reservoir 614 under the force of gravity indicated by arrow 693. In this manner, contaminant-containing liquid is collected continuously during the operation of apparatus 601.

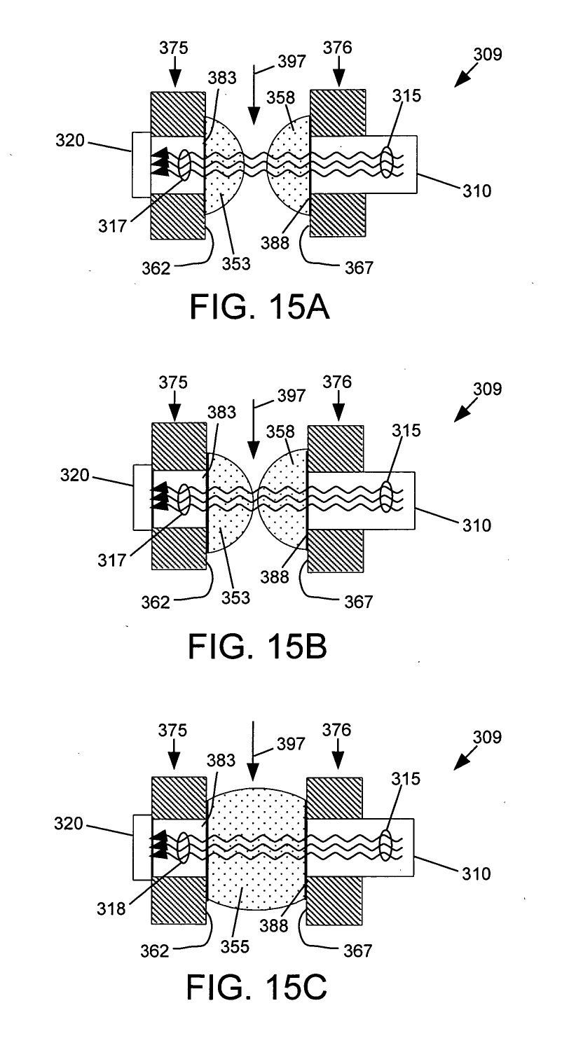

FIGS. 15A-15C are cross-sectional sequential illustrations of the bridging of a gap by a liquid drop within a detection channel in an embodiment of the applicants' apparatus further comprising hydrophilic surfaces that include reactive species. Referring to FIG. 15A, apparatus 309 is similar in structure to the apparatus 300 of FIG. 9, and comprises walls 375 and 376 with hydrophobic surfaces 362 and 367, light source 310, and optical sensor 320. Wall 375 further comprises a hydrophilic dot 383 containing a first chemical species C₁, and wall 376 further comprises a hydrophilic dot 388 containing a second chemical species C₂.

Referring also to FIGS. 15B and 15C, as droplets 353 and 358 condense onto dots 383 and 388, such droplets grow as indicated in FIG. 15B, eventually touching and coalescing into droplet 355 as indicated in FIG. 15C. During droplet growth in FIGS. 15A and 15B, light 315 from light source 310 passes through droplets 358 and 353, and attenuated light 317 is received by optical sensor 320. Reactive species C1 and reactive species C2 dissolve into droplets 358 and 353 during droplet growth. When droplets 358 and 353 coalesce to form droplet 355 as indicated in FIG. 15C, reactive species C3 is formed. Such reactive species C3 is selected to be further able to react with one or more contaminants in air stream 397, thereby providing attenuated light 318, which is detectable by optical sensor 320. Thus contaminants in air stream 397 are detectable by the apparatus 309. In a further embodiment (not shown), the formation of drop 355 can also be detected by applying a voltage to dots 383 and 388, and detecting the coalescence by a flow of electric current in the circuit

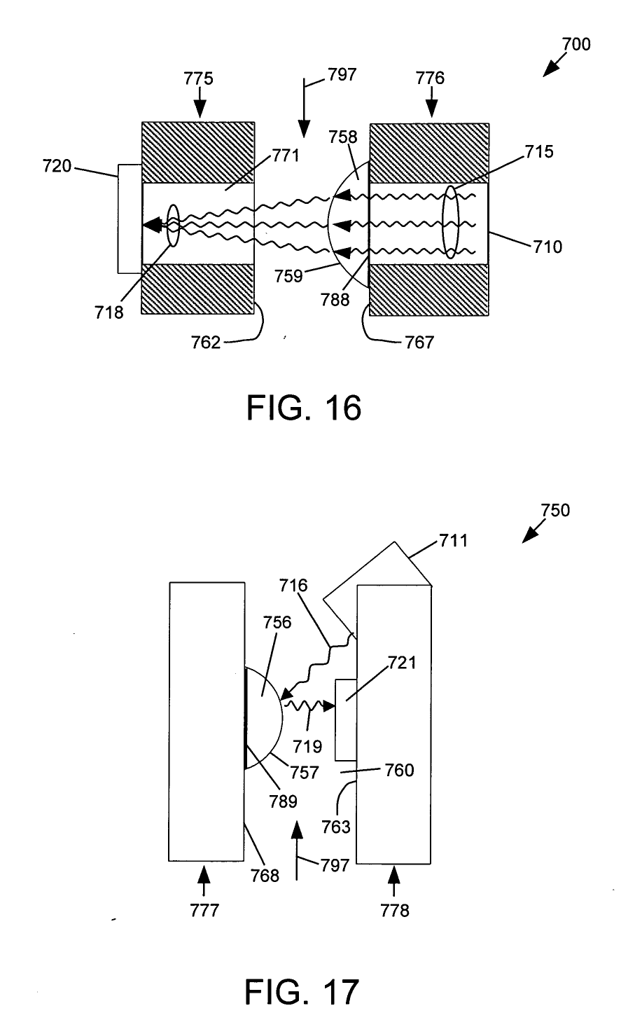

FIG. 16 is a cross-sectional illustration of a further embodiment of the applicant's apparatus wherein light transmitted through a droplet is focused by the meniscus of such drop within a detection chamber. Referring to FIG. 16, apparatus 700 comprises wall 776 which includes light source 710, hydrophobic surface 767, hydrophilic dot 788; and wall 775 which includes optical sensor 720, transparent window 771, and hydrophobic surface 762. In operation, drop 758 is formed by condensation of saturated moisture from flowing air stream 797 due to the combination of the hydrophilic “seed” dot coating 788 and hydrophobic surrounding surface 767. Meniscus 759 of drop 758 serves as an optical lens for light 715 from light source 710, providing focused light 718 on optical sensor 720. The optical absorbance of specific wave lengths in the liquid drop 758, which is dependent upon the type and concentration of contaminants in drop 758, can thus be measured. Contaminant materials which are adsorbed at the meniscus 759, or absorbed into the bulk of the droplet can both be detected.

FIG. 17 is a cross-sectional illustration of a further embodiment of the applicant's apparatus wherein light is reflected or scattered from the meniscus of a drop to an optical sensor within a detection chamber. Referring to FIG. 17, apparatus 750 comprises light source 711, wall 777 which includes hydrophobic surface 768, hydrophilic dot 789; and wall 778 which includes optical sensor 721, and hydrophobic surface 763. In operation, drop 756 is formed by condensation of saturated moisture from flowing air stream 797 due to the combination of the hydrophilic “seed” dot coating 789 and hydrophobic surrounding surface 768. Meniscus 757 of drop 756 serves as an optical reflector, reflecting incoming light 715 from light source 710, providing reflected light 719 on optical sensor 721. The optical absorbance of specific wave lengths in the liquid drop 758, which is dependent upon the type and concentration of contaminants in drop 756, can thus be measured. Light with specified wavelengths can be directed to drop 756. The scattered/reflected light 719 is detected for density or optical spectral analysis by optical sensor 721.

In one embodiment, the solid surface of dot 789 and/or hydrophilic surface 768 can be curved. For example, a cylindrical or spherical shaped solid surface can be used to maintain the liquid drop and to optimally arrange the light source 711 and optical sensor 721 for optical detection. It will be apparent that although optical sensor 721 is depicted as being disposed within chamber 760 on the inner surface 763 of wall 778, an optically transparent window can be provided in wall 778 so that optical sensor can be disposed on the exterior of wall 778, as was depicted and previously described herein for optical sensor 720 of FIG. 16.

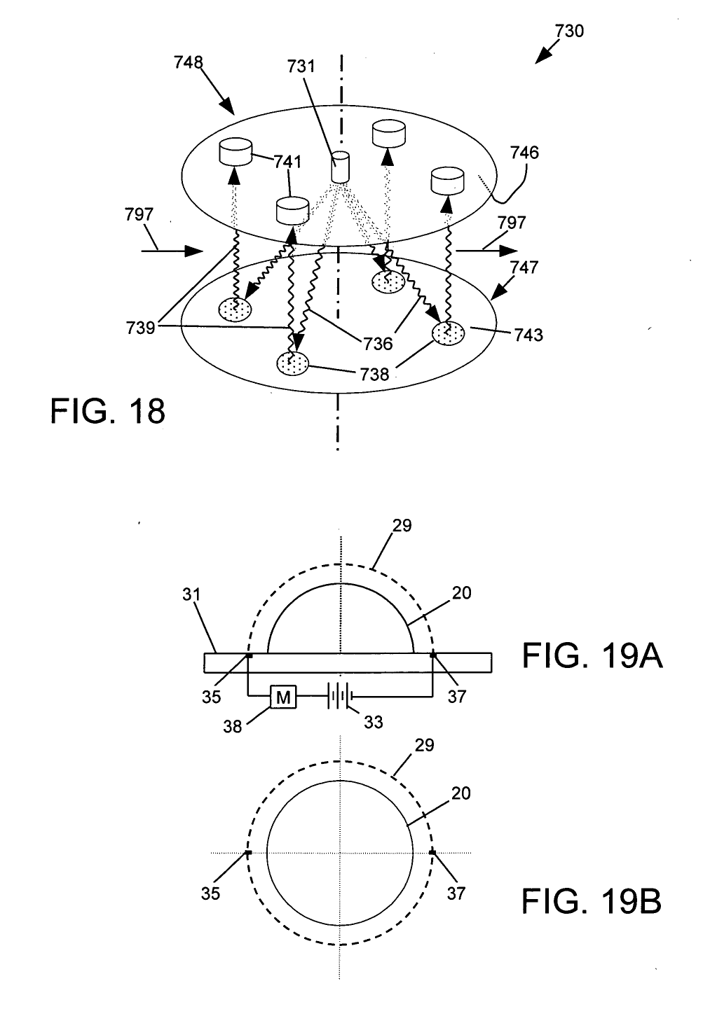

FIG. 18 is a simplified perspective view of a multi-drop embodiment of the collection and detection apparatus of FIG. 17. Referring to FIG. 18, apparatus 730 comprises a first chamber wall 748 comprising hydrophobic surface 746 and a second chamber wall 747 comprising hydrophobic surface 743, upon which are disposed hydrophilic dots (not shown), upon which in turn, droplets 738 condense during the operation of apparatus 730. First chamber wall 748 further comprises at least one light source 731, which directs light 736 upon droplets 738 through a transparent window in such wall 748. Droplets 738 reflect light as described previously herein through additional transparent windows (not shown) to optical sensors 741, which are disposed on first chamber wall 748.

In operation, a contaminant containing air stream indicated by arrows 797 is directed between surfaces 743 and 746, and contaminants are captured by droplets 738 and optically analyzed as previously described herein. It will be apparent that apparatus 730 may comprise many more drops 738, optical sensors 741, and light sources 731, as required for the detection and analysis of many potential contaminants in the air stream. Additionally, it is not required that the solid surfaces 746 and 743 are flat. Apparatus 730 may be configured in a cylindrical shape wherein the detection chamber is annular, or a spherical shape wherein the detection chamber is a spherical shell, or in a parabolic or other curvilinear shape.

FIGS. 19A and 19B are side and top views, respectively, of means to detect the size of a drop by detecting electrical resistance change when such drop wets a substrate coated with conductive material. Referring to FIGS. 19A and 19B, drop 20 is condensed or otherwise deposited upon surface 31. If drop 20 is condensed to an undesirably large sized drop 29, the contact line of drop 29 expands outwardly such that drop 29 wets a pair of contacts 35 and 37 provided in surface 31 (or alternatively, a conductive ring in surface 31), which are connected to electric power supply 33. Drop 29 then either conducts electrical current or becomes capacitively charge, depending upon its properties. In either case, the contact of drop 29 with contacts 35 and 37 is detectable by a suitable meter 38 or other detection means, indicating that drop 29 has grown undesirably large. The temperature of the detection system, or the dew point of the flowing air stream may then be adjusted to provide drops of the desired size.

FIG. 20A is a side cross-sectional view of another multi-channel collection and detection apparatus of the present invention; and FIG. 20B is a top cross sectional view of the apparatus of FIG. 20A taken along the line 20B-20B of FIG. 20A. Referring to FIGS. 20A and 20B, there is provided a first channel 833 between walls 832 and 834, within which flows contaminant containing liquid as indicated by arrow 898. Wall 834 is further provided with a hydrophobic surface 837 with small holes 835 therein, wherein liquid menisci 820 are formed at the outer ends thereof. Due to the effect of surface tension, these menisci will provide a backpressure to resist flowing of fluid out of the channels 835. In general, the smaller the hole diameter, the larger the back pressure the menisci can create. The holes 820 may be cylindrical, instead of tapered as shown in FIG. 20A. Holes 820 preferably have a diameter of less than about 1 micrometer, although larger holes may function satisfactorily, depending upon the surface tension of the contaminant-capturing liquid.

Referring again to FIG. 20A, a contaminant-containing air stream flows as indicated by arrow 897 through a second channel formed by wall 834 and wall 836. Contaminants are collected in the liquid at menisci 820 and are detected by optical sensors 810, which are disposed on wall 836. For the sake of simplicity of illustration, a light source is not shown in FIGS. 20A and 20B; however, such light source is provided in either the reflective mode or the transmissive mode as described previously in this specification. Referring to FIG. 20B, and in the preferred embodiment, multiple rows 841, 842, and 843 of channels are provided. One advantage of this embodiment is that a relatively large number of liquid drops can be created on a small area of solid surface. Another advantage of this embodiment is that many different types of liquid drops (e.g. hydrophobic and hydrophilic, containing different chemicals, carrying positive or negative charge, etc.) can be created on a small area of a solid surface.

FIG. 21 is a perspective sectional view of a cylindrical embodiment of the multi-channel collection and detection apparatus of FIGS. 20A and 20B. Referring to FIG. 21, apparatus 850 comprises a cylindrical shell 884, through which are provided axial channels 883. Along axial channels 883, holes 885 are formed, connecting the inner surface 887 to channels 883. Inner surface 887 is a hydrophobic surface, such that when contaminant containing liquid is delivered through channels 883 as indicated by arrows 898, menisci 870 are formed on inner surface 887.

In operation, a contaminant containing air stream is delivered through chamber 860 as indicated by arrow 897. Contaminants are collected at menisci 870, and are analyzed by optical detection means. For the sake of simplicity of illustration, such optical detection means are not shown, but may be configured in the transmissive or reflective modes as described previously in this specification. Alternatively, flow through channels 883 may be made intermittent, such that the contaminant contents of menisci 870 are withdrawn and analyzed in a collection device (not shown) that is downstream of apparatus 850.

Referring again to FIG. 21, channels 883 may be interconnected and deliver a single liquid therethrough, or channels 883 may be isolated so that a different collection liquid may be used in each channel 883. As was indicated previously, surface 887 of shell 884 is hydrophobic if a hydrophilic collection liquid is used; similarly, surface 887 of shell 884 is hydrophilic if a hydrophobic collection liquid is used. In one embodiment, part of surface 887 can be made hydrophilic and part of the surface can be made hydrophobic such that both hydrophobic and hydrophilic liquids may used in different channels 883. The inside of a channel 883 is always made wettable to the particular liquid in that channel.

It is, therefore, apparent that there has been provided, in accordance with the present invention, a method and apparatus for airborne chemical and biological sample collection and detection in real time using optical or chemical techniques. While this invention has been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of the appended claims.

I claim:

1. An apparatus for analysis of a flowing gas stream containing a substance, said apparatus comprising a chamber for delivery of said flowing gas stream containing said substance, said chamber comprising a first wall including a wall surface and a first channel disposed within said first wall and terminating at an outlet edge at said wall surface, said channel provided for delivering a liquid to said chamber such that a gas-liquid interface is formed and maintained in a fixed position in said chamber at said outlet edge of said channel.

2. The apparatus as recited in claim 1, wherein said wall surface is a hydrophobic surface, and said channel is comprised of a hydrophilic surface, such that a transition from a hydrophobic surface to a hydrophilic surface occurs at said outlet edge.

3. The apparatus as recited in claim 1, further comprising a light source and an optical sensor.

4. The apparatus as recited in claim 1, further comprising a plurality of channels disposed within said wall, each of said channels terminating at an outlet edge at said wall surface, and each of said channels provided for delivering a liquid to said chamber such that a gas-liquid interface is formed and maintained in a fixed position in said chamber at said outlet edges of said channels.

5. The apparatus as recited in claim 4, wherein said plurality of channels is a rectangular array of channels.

6. The apparatus as recited in claim 4, wherein said wall surface of said first wall is a cylinder.

7. The apparatus as recited in claim 1, further comprising a liquid disposed in said channel, said liquid bounded by a gas liquid interface formed at said outlet edge of said channel.

8. An apparatus for analysis of a flowing gas stream containing a substance, said apparatus comprising a chamber for delivery of said flowing gas stream containing said substance, said chamber comprising:
a. a first wall including a first wall surface and a longitudinal inlet channel disposed within said first wall and terminating at an outlet edge at said first wall surface;
b. a second wall including a second wall surface and a longitudinal outlet channel disposed within said second wall and beginning at an outlet edge at said second wall surface, said longitudinal inlet channel aligned with said longitudinal outlet channel, and said first wall surface separated from said second wall surface by a gap;
wherein said longitudinal inlet channel and said longitudinal outlet channel are adapted for delivering a liquid into said inlet channel, through said chamber, and out said outlet channel such that a gas-liquid interface may be formed and maintained in a fixed position in said gap of said chamber.

9. The apparatus as recited in claim 8, wherein said first and second wall surfaces are hydrophobic surfaces, and said first and second channels are comprised of hydrophilic surfaces, such that a transition from a hydrophobic surface to a hydrophilic surface occurs at said outlet edge of said first channel and said outlet edge of said second channel.

10. The apparatus as recited in claim 8, further comprising a light source and an optical sensor.

11. The apparatus as recited in claim 8, further comprising a plurality of longitudinal inlet channels disposed within said first wall, each of said inlet channels terminating at an outlet edge at said first wall surface, a plurality of longitudinal outlet channels disposed within said second wall, each of said outlet channels beginning at an outlet edge at said second wall surface, wherein each of said inlet channels is aligned with one of said outlet channels.

12. The apparatus as recited in claim 11, wherein said plurality of inlet channels and outlet channels is a rectangular array of channels.

13. The apparatus as recited in claim 11, wherein said first and second wall surfaces are cylindrical surfaces forming an annulus therebetween.

14. The apparatus as recited in claim 8, further comprising a liquid disposed in said gap, said liquid bounded by an air liquid interface formed between said first wall surface and said second wall surface.

15. An apparatus for analysis of a flowing gas stream containing a substance, said apparatus comprising a first wall including a first hydrophobic surface upon which is disposed a first hydrophilic surface; and a second wall including a second hydrophobic surface upon which is disposed a second hydrophilic surface, said first hydrophilic surface aligned with said second hydrophilic surface, and said first hydrophilic surface separated from said second hydrophilic surface by a gap.

16. The apparatus as recited in claim 15, further comprising means for cooling said first hydrophobic surface and said second hydrophobic surface.

17. The apparatus as recited in claim 15, further comprising a light source and an optical sensor.

18. The apparatus as recited in claim 15, further comprising a plurality of hydrophilic surfaces on said first hydrophobic surface of said first wall and a plurality of hydrophilic surfaces on said second hydrophobic surface of said second wall, wherein each of said hydrophilic surfaces on said first wall is aligned with a hydrophilic surface of said second wall.

19. The apparatus as recited in claim 18, wherein said plurality of hydrophilic surfaces on said first hydrophobic surface of said first wall and said plurality of hydrophilic surfaces on said second hydrophobic surface of said second wall is a rectangular array.

20. The apparatus as recited in claim 15, wherein said first and second hydrophobic surfaces are cylindrical surfaces forming an annulus therebetween.

21. The apparatus as recited in claim 15, wherein said first hydrophilic surface comprises a first chemical species and said second hydrophilic surface comprises a second chemical species, and said first chemical species is reactive with said second chemical species.

22. The apparatus as recited in claim 15, further comprising a liquid droplet disposed on at least one of said first and said second hydrophilic surfaces, said liquid drop forming a gas-liquid interface with said flowing gas stream.

23. A method for using liquid drops to capture gas borne substances in a flowing gas stream, said method comprising the steps of:
a. providing an apparatus comprising a chamber for delivery of said flowing gas stream containing said substance, said chamber comprising a first wall including a first wall surface, a second wall including a second wall surface separated from said first wall surface by a gap;
b. causing said flowing gas stream to flow though said chamber;
c. providing a liquid that forms at least one gas-liquid interface in said chamber in said gap between said first wall surface and said second wall surface;
d. detecting at least one substance in said liquid that has passed from said flowing gas stream through said gas-liquid interface into said liquid.

24. The method as recited in claim 23, wherein said apparatus further comprises a longitudinal inlet channel in said first wall, and a longitudinal outlet channel in said second wall, and said gas-liquid interface is formed in said gap and between said longitudinal inlet channel, and said longitudinal outlet channel.

25. The method as recited in claim 23, wherein said first wall includes a first hydrophobic surface upon which is disposed a first hydrophilic surface, and said second wall includes a second hydrophobic surface upon which is disposed a second hydrophilic surface, and said gas-liquid interface is formed in said gap and between said first hydrophilic surface, and said second hydrophilic surface.

26. The method as recited in claim 23, wherein said flowing gas stream is an air stream.

27. The method as recited in claim 26, wherein said substance is selected from the group consisting of gaseous, solid, liquid, aerosol, organic, inorganic, hydrophobic, hydrophilic, radioactive, living, and non-living substances.

28. A method for using a liquid to capture gas borne substances in a flowing gas stream, said method comprising the steps of:
a. exposing at least part of said liquid to said gas stream to form a gas-liquid interface;
b. limiting the area of each said gas-liquid interface so that said gas-liquid interface is stabilized by surface tension; and
c. maintaining said gas-liquid interface in a fixed position on a solid surface.
